(19) United States
(12) Patent Application Publication
(10) Pub. No.: US 2007/0020162 A1
(43) Pub. Date: Jan. 25, 2007

(54) Sterilization heating module

(75) Inventors: James A. Scudder, Poway, CA (US); Peter J. Kapitzke, Poway, CA (US); Kenneth M. Sanderman, Lakeside, CA (US)

Correspondence Address: Vista IP Law Group LLP, 2040 MAIN STREET, 9TH FLOOR, IRVINE, CA, 92614, US

(73) Assignee: OnTech Delaware Inc.

(21) Appl. No.: 11/186,463

(22) Filed: Jul. 20, 2005

(51) Int. Cl. A61L 2/04

(52) U.S. Cl. 422/307; 422/232

(57) ABSTRACT

A heating module for use with a sterilizer utilizes an exothermic chemical reaction produced by mixing a first reactant with a second reactant to produce heat without a flame or combustion. The heating module may comprise a pouch filled with a first reactant. The pouch is made of a porous material or fabric such as a nylon or polyester woven material. The pores in the material are small enough contain the particulate or powdered solid reactant within the pouch but are large enough to allow a second reactant to permeate the pouch. The pouch may be placed directly into the water reservoir of a sterilizer. The second reactant is then added to the water reservoir and permeates the pouch thereby activating the exothermic reaction. The heat from the reaction vaporizes the water into high temperature, high pressure steam. The heating module may alternatively comprise a first reactant compartment and a second reactant compartment. The first reactant compartment contains a first reactant that is physically separated from a second reactant contained in the second reactant chamber. When a user actuates the thermic module, the reactants mix and produce an exothermic reaction to heat water into high temperature, high pressure steam.

### FIELD OF THE INVENTION

The present invention relates generally to portable sterilizers for sterilizing medical instruments, and more particularly to a non-electrical heat source for such sterilizers.

### BACKGROUND OF THE INVENTION

Medical instruments must be sterilized to neutralize bacteria and other microbials that can cause infections in the patients on which the instruments are used. Medical instruments are most commonly sterilized in a device called an autoclave. An autoclave typically includes a thermally insulated pressure chamber in which the medical instruments are subjected to high temperatures and pressures for a period of time. The size of the chamber can vary widely depending on the capacity needed for the particular application. For example, some autoclaves can occupy an entire room, whereas portable autoclaves may fit on a desk top or cabinet. The chamber may also include one or more racks or shelves for placing the instruments, or a tray of instruments, on during the sterilization process. A heat source and a supply of water are provided to produce steam in the chamber. The chamber is sealed to withstand the significant pressures created by the hot steam. The high temperature and pressure kills the bacteria and microbials which may be present on the instruments being sterilized.

The most common heat source used with autoclaves is an electrical heater. Still, some portable sterilizers are designed without an electric heat source and instead are used with a separate heat source such as a stove or a Bunsen burner. The primary advantages of a non-electric heat source are that the sterilizer can be completely self-contained without the need for a source of electricity. There are a number of commercially available non-electric sterilizer designed for use with a stove or Bunsen burner. These portable sterilizers generally have a water reservoir such as a tray or basin and the heat from the stove or Bunsen burner is applied to the water reservoir to produce high temperature, high pressure steam. These sterilizers are extremely useful in situations and locations where there is not a convenient source of electricity, such as in rural areas, in military uses and veterinary uses and during power outages.

While Bunsen burners and gas stoves are a relatively good source of portable heat, they have several drawbacks. First, burners and stoves use a flame and therefore present a fire hazard. In addition, portable burners and stoves require tanks of gas which can be heavy and are also somewhat fragile and potentially volatile. In addition, burners and stoves must be lit with match, lighter or pilot.

Accordingly, the present invention is directed to a sterilizer heating module which is particularly useful in portable sterilizers and which overcomes some of the problems and deficiencies of currently available sterilizer heat sources.

### SUMMARY OF THE INVENTION

The sterilization heating module of the present invention may be used with any suitable sterilizer but is especially useful for portable sterilizers. The heating module of the present invention is portable, lightweight, flameless and does not need to be lit.

The heating module utilizes an exothermic reaction to produce heat in order to heat water thereby producing steam. The exothermic reaction is produced by combining a first reactant with a second reactant. The first reactant preferably comprises a solid compound in a granular or powder form. The second reactant is preferably a liquid and more preferably water because it is readily available and also because the portable sterilizers utilize steam to accomplish the sterilization process. Calcium oxide (commonly known as limestone) and water are examples of two reactants known to produce an exothermic reaction wherein one of the reactants is a liquid (water) and the other reactant (calcium oxide) may be easily obtained in a granular or powder form.

In one embodiment of the present invention, the heating module may simply comprise a pouch filled with the first reactant. The pouch is made of a porous material or fabric such as a nylon or polyester woven material. The pores in the material are small enough contain the particulate or powdered solid reactant within the pouch but are large enough to allow the second reactant to permeate the pouch. The pouch may be packaged within a moisture proof package so that the first reactant within the pouch is protected from water, including humidity in the air. The use of this heating module is simple and straightforward. The pouch may be placed directly into the water reservoir of the sterilizer. Then, water is dispensed into the water reservoir. The water permeates the pouch thereby activating the exothermic reaction. The heat from the reaction vaporizes the water into high temperature, high pressure steam.

In another embodiment of the present invention, the heating module may be configured to contain both the first reactant and the second reactant in a self-contained unit. The module comprises a first reactant compartment and a second reactant compartment. The first reactant compartment contains a first reactant that is physically separated from a second reactant contained in the second reactant chamber. When a user actuates the thermic module, the reactants mix and produce an exothermic reaction to heat water into high temperature, high pressure steam. To use the module in the sterilizer, the heating module is actuated to cause the reactants to mix. The entire module is then placed into the water reservoir of the sterilizer. Additional water may be added to the water reservoir before or after inserting the heating module, depending on the configuration of the heating module and the requirements of the particular sterilizer.

The foregoing, together with other features and advantages of the present invention, will become more apparent when referring to the following specification, claims, and accompanying drawings.

### BRIEF DESCRIPTION OF THE DRAWINGS

For a more complete understanding of the present invention, reference is now made to the following detailed description of the embodiments illustrated in the accompanying drawings, wherein:

FIG. 1 is a partial cut-away, perspective view of a heating module according to a first embodiment of the present invention;

FIG. 2 is a cross-sectional side view of a heating module according to second embodiment of the present invention;

FIG. 3 is a cross-sectional top view of the heating module of FIG. 2.

### DESCRIPTION OF PREFERRED EMBODIMENTS

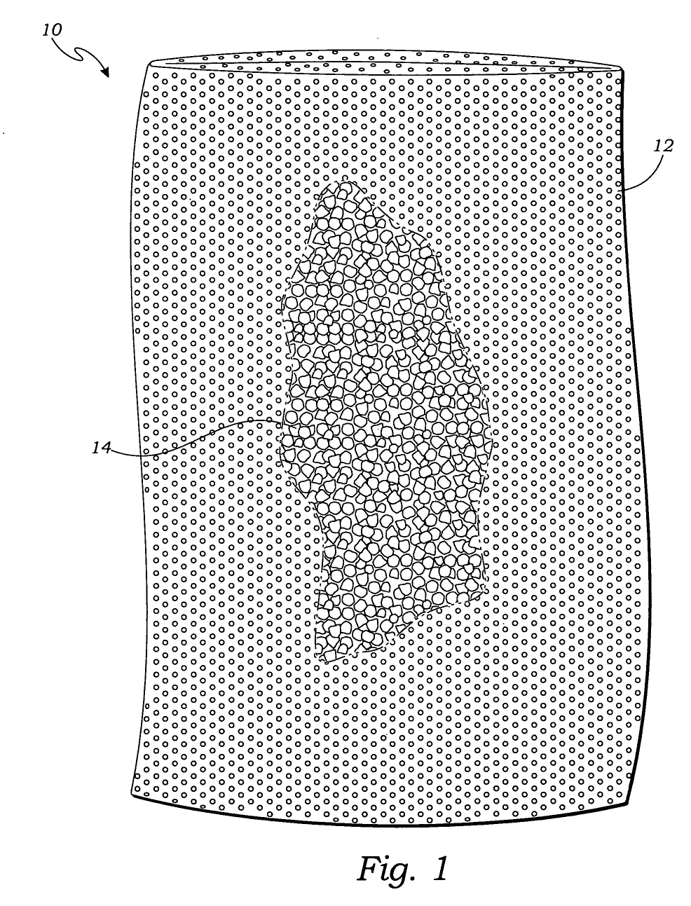

Turning to FIG. 1, a heating module 10 for use with sterilizers is shown. The heating module 10 comprises a pouch 12. The pouch 12 contains a first reactant 14 which is preferably in a solid granular or powder form, such as calcium oxide. It should be understood that other compounds may be utilized as the first reactant 14 within the present invention, so long as such compound can be mixed with another reactant to produce an exothermic reaction. The first reactant 14 preferably reacts with a second reactant (not shown) which can easily be obtained in a liquid form, such as water, to produce an exothermic reaction. The second reactant may also be a gas or a very fine powder, so long as it can permeate the pouch and react with the first reactant to produce the necessary exothermic reaction. Those skilled in the art, in view of the present teachings, will be able to determine first and second reactants and their physical form which can be used in the present invention.

The pouch 12 is made of a porous material which can effectively contain the first reactant 14 in a granular or powder form within the pouch. For example, the pouch may be made of woven nylon, polyester or any other suitable fabric or film. Effective containment can vary from completely preventing the first reactant 14 from penetrating the material, to allowing a reasonable amount of the smaller particles or the first reactant 14 to leak through the material but containing the majority of the first reactant 14. In addition, the pores in the pouch 12 material must be large enough that the second reactant can permeate the pouch 12 and react with the first reactant 14. If the first reactant 14 reacts with air or moisture, the pouch 12 may be packaged in an airtight or moisture proof package (not shown) to protect the first reactant.

The use of the heating module 10 with a sterilizer having a waters reservoir is as follows. The heating module 10 is placed directly into the water reservoir. Then, the second reactant is dispensed into the water reservoir. Alternatively, the second reactant may be dispensed into the water reservoir prior to placing the heating module into the water reservoir. Where the second reactant is other than water, water is dispensed into the water reservoir at some point during the process. For example, the water could be dispensed into the reservoir prior to placing the pouch into the water reservoir or it could be dispensed after the heating module 10 and second reactant are placed into the water reservoir. The second reactant then permeates the pouch 12 and mixes with the first reactant 14 thereby activating the exothermic reaction. The heat from the reaction vaporizes the water into high temperature, high pressure steam.

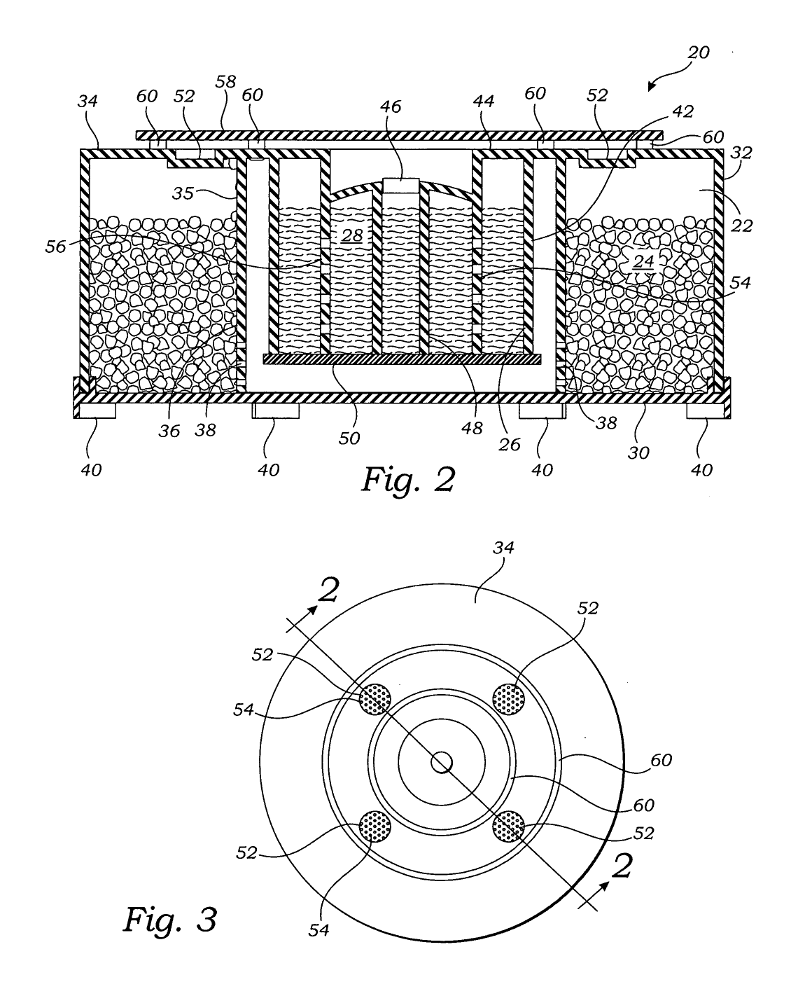

Another heating module 20 according the present invention is shown in FIGS. 2 and 3. The heating module 20 is similar to the heating module 10 in that it utilizes an exothermic reaction to produce heat. The heating module 20 comprises a first compartment 22 containing a first reactant 24 and a second compartment 26 containing a second reactant 28. The heating module 20 may have a round shape as shown, or it may have a square, rectangular, elliptical or any other suitable shape. The first reactant 24 is preferably a solid granule or powdered reactant and the second reactant 28 is a liquid. For example, the types of first reactant 14 and second reactant described above with respect to the heating module 10 are equally suitable for first reactant 24 and second reactant 28, respectively.

The first compartment 22 comprises a bottom 30, an outer sidewall 32 extending upward from the bottom 30, an inner sidewall 36 extending upward from the bottom 30, and a top 34. The bottom 30, sidewalls 32 and 35, and top 34 form a substantially enclosed annular chamber for containing the first reactant 24. The first compartment 22 may be formed of one or more pieces of molded polypropylene, or other suitable plastic. The bottom portion of the inner sidewall 36 has a plurality of holes or slits 38 which allow the second reactant 28 to enter the first compartment 22 when the heating module 20 is actuated. A plurality of feet 40 for supporting the heating module 20 when placed on a flat surface.

The top 34 of the first compartment 22 has a plurality of vent holes 52 which each have a cover 54. The covers 54 allow steam to pass through it but at least substantially prevents the solid first reactant 24 from escaping the first compartment. The cover 54 may be made of the same or similar material as that described above for the pouch 12. Alternatively, the covers 54 may simply be very small perforations or holes in the top 34.

The second compartment 26 comprises a cylindrical chamber formed by an outer wall 42, and a flexible cap 44. A breakable reactant barrier 50, preferably made of a metal foil seals the open lower end of the cylindrical chamber formed by the outer wall 42 and the flexible cap 44. The reactant barrier 50 may be attached to the open end of outer wall 42 by thermal bonding, ultrasonic bonding, use of an adhesive or any other suitable method. The flexible cap 44 has a disc-shaped or dome shaped actuator button 46 and a cylindrical prong 48. The cylindrical prong 48 extends toward the breakable barrier 50. Additional prongs 48 may be added depending on the size of the breakable barrier 50. For example, another cylindrical actuation prong 54 may be provided which surrounds the first prong 48. The second prong 54 may have a plurality of slits or holes 56 so that when the second reactant 28 is dispensed into the compartment 26 it flows through the holes 56 to fill the entire compartment 26.

The second compartment 26 may be formed integrally with the first compartment 22 or it may be attached thereto by an adhesive, by ultrasonic or spin welding or by any other suitable method.

A tamper-evident seal 58 is attached to the top 34 and covers both the actuator button 46 and the holes 52 in the top 34 such that the seal 58 must be removed or damaged to actuate the button 46. The tamper-evident seal 58 may be a foil decal adhesively attached to raised ribs 60 formed on the top 34.

The use of the heating module 20 with a sterilizer (not shown) having a water reservoir is as follows. First, the user removes the tamper-evident seal 58 to expose the actuator button 46. The user depresses the actuator button 46 by pushing it inward. The force exerted upon the actuator button 46 moves the prongs 48 and 56 into the reactant barrier 50. The prongs 48 and 56 puncture the reactant barrier 50 which allows the second reactant 28 in the second compartment 26 to flow through the holes 38 into the first compartment 22. The second reactant 28 mixes with first reactant 24 thereby creating an exothermic reaction. The resulting exothermic reaction produces heat, which vaporizes the second reactant 28.

The heating module 20 is then placed into the water reservoir of the sterilizer. In the case where the second reactant 28 is water, the steam created by the reaction flows upward. through the holes 52 and into the water reservoir. The hot steam is used by the sterilizer to create high temperature, high pressure steam which sterilizes the objects placed in the sterilizing chamber of the sterilizer.

In addition, water may be added to the water reservoir surrounding the heating module 20. The outside surfaces of the second compartment 22, including the outer sidewall 32 and the bottom 30, may be designed to become hot during the exothermic reaction. In such case, the outside surfaces of the second compartment 22 will heat the water in the water reservoir, thereby creating more high temperature, high pressure steam. In this case, it is not necessary the second reactant 28 be water because the steam will be produced outside of the exothermic reaction occurring within the first compartment 22. In order to enhance the heat transfer between the second compartment and the surrounding water, the outside surfaces may be corrugated, wavy and/or ribbed to increase their surface area. Moreover, the heating module 20 may further comprise another protective wall 62 (not shown) which is attached to and surrounds the second compartment 22 but which leaves an air space 64 (not shown) between the protective wall 62 and the outside surfaces of the second compartment 22. The protective wall 62 prevents a user from touching the hot surfaces of the second compartment 22 but allows water to flow into the air space 64 such that the water in a water reservoir is heated into high temperature, high pressure steam.

While the present invention has been fully described above with particularity and detail in connection with what is presently deemed to be the invention, it will be apparent to those of ordinary skill in the art that many modifications thereof may be made without departing from the principles and concepts set forth herein. Hence, the invention, should not be limited, except to the following claims, and their equivalents.

What is claimed is:

1. A heating module for use with a sterilizer, comprising:
a container made of a porous material and containing a first reactant, said porous material having pores which are small enough to substantially contain said first reactant within said container but large enough to allow a second reactant to permeate the material; and
wherein said first reactant and said second reactant produce an exothermic reaction when mixed together.

2. The heating module of claim 1 further comprising a moisture proof package covering said container.

3. The heating module of claim 1 wherein said first reactant is calcium oxide and said second reactant is water.

4. A method of using a sterilizer comprising a water reservoir, comprising the following steps:
inserting a heating module into the water reservoir of the sterilizer, said heating module comprising a container made of a porous material and containing a first reactant, said porous material having pores which are small enough to substantially contain said first reactant within said container but large enough to allow a second reactant to permeate the material;
dispensing said second reactant into said water reservoir; and
dispensing water into said water reservoir.

5. The method of claim 4 wherein said water is dispensed into said water reservoir after said heating module is inserted into said water reservoir.

6. The method of claim 4 wherein said water is dispensed into said water reservoir before said heating module is inserted into said water reservoir.

7. The method of claim 4 wherein said second reactant is dispensed into said water reservoir after said heating module is inserted into said water reservoir.

8. The method of claim 4 wherein said second reactant is dispensed into said water reservoir before said heating module is inserted into said water reservoir.

9. The method of claim 4 wherein said second reactant is water.

10. The method of claim 4 wherein said first reactant is calcium oxide and said second reactant is water.

11. A heating module for use with a sterilizer, comprising:
a first reactant chamber containing a first reactant, said first reactant chamber having a plurality of vent holes having covers which substantially contain said first reactant in said first reactant chamber but allow vapor to pass through said hole;
a second reactant chamber containing said second reactant, said first and second reactant chambers separated from one another by a breakable barrier;
an actuator which breaks said breakable barrier when actuated to allow mixing of said first and second reactants.

12. The heating module of claim 111 further comprising a tamper-evident seal attached to said first reactant chamber, said seal covering said actuator and said holes.

13. A heating module for use with a sterilizer, comprising:
a container having a chamber containing a first reactant, said chamber having an inlet for allowing a second reactant to enter said chamber; and
wherein said first reactant and said second reactant produce an exothermic reaction when mixed together.

14. The heating module of claim 13 wherein said inlet comprises a hole through which the second reactant is dispensed into said chamber.

15. The heating module of claim 13 wherein said inlet is a plurality of holes through which a second reactant enters said chamber.

16. A method of using a sterilizer comprising a water reservoir, comprising the following steps:
inserting a heating module into the water reservoir of the sterilizer, said heating module comprising a container having a chamber containing a first reactant, said chamber having an inlet for allowing a second reactant to enter said chamber; and
wherein said first reactant and said second reactant produce an exothermic reaction when mixed together;
mixing said first reactant with said second reactant; and
dispensing water into said water reservoir.

17. The method of claim 16 wherein said step of mixing said first reactant with said second reactant is performed prior to said step of inserting said heating module into said water reservoir.

18. The method of claim 16 wherein said step of mixing said first reactant with said second reactant is performed after said step of inserting said heating module into said water reservoir.
